(19) United States
(12) Patent Application Publication
(10) Pub. No.: US 2004/0022748 A1
(43) Pub. Date: Feb. 5, 2004

(54) Method of enhancing skin lightening

(75) Inventors: Kavssery Parameswaran Ananthapadmanabhan, Highland Mills, NY (US); Gail Beth Rattinger, Teaneck, NJ (US); William Becker, Rockaway, NJ (US); Yuet Tong, Fort Lee, NJ (US); Andrew Lam, Yorktown, NY (US); Rajendra M. Dave, Newark, NJ (US); Eng-Beng Lim, Jakarta (ID)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/095,658

(22) Filed: Mar. 12, 2002

(51) Int. Cl. A61K 7/04; A61K 7/135

(52) U.S. Cl. 424/61

(57) ABSTRACT

The present invention discloses a method or process for enhancing skin lightening of one wash-off product relative to another wherein said method or process comprises using a composition which is less harsh (measured by zein solubility) relative to the other.

### FIELD OF THE INVENTION

The present invention relates to personal product cleanser compositions (e.g., which may be delivered in the form of bars, liquids, non-wovens). In particular, it relates to such personal product cleanser compositions which provide measured skin lightening effects as perceived by trained evaluators or tested subjects themselves. Applicants are unaware of any art linking the degree of mildness of personal product composition (e.g., liquid, personal wash cleansing compositions), to their skin lightening effects.

### BACKGROUND

It is a general desire among many consumers to utilize products which have a skin lightening effect. The general belief in the art in this regard has been that skin lightening requires specific actives that interfere with the biochemistry of melanin, i.e., a brownish-black pigment found in the skin which is normally associated with skin darkening.

In the subject invention, applicants have found that the use of certain soaps in personal wash compositions, in particular harsher fatty acid soaps, can lead to hyper-pigmentation effects. Thus, applicants have hypothesized and subsequently shown that, by minimizing the effect of such harsh cleansers, it is possible to simultaneously enhance skin lightening relative to a separate skin cleanser which is relatively harsher. Harshness of compositions is determined using zein solubility measurements (e.g., generally the harsher the product, the more zein it will dissolve).

### BRIEF DESCRIPTION OF THE INVENTION

The present invention provides a method for enhancing the skin lightening (e.g., perceived and assessed by self-assessment and/or by a specially trained evaluator) of a wash-off, personal product composition (preferably a cleansing composition comprising one or more surfactants) relative to a second wash-off personal product composition wherein the process comprises applying or utilizing said first composition such that it has a zein solubility for said first composition of about 2% or less (as measured by specified zein test) and wherein said first composition is compared/tested against a second composition which has a zein solubility, in relative terms, greater than that of the first composition and, preferably, greater than about 3% in absolute terms (again as measured by specified test). Zein solubility is a measure of the harshness of the compositions (for example, soap is generally harsher than most synthetic surfactants) and zein solubility measurements are taken as defined in the protocol section of the specification below. The compositions may be delivered in the form of liquids, bars, non-woven substrates comprising the compositions etc.

In a separate embodiment of the invention, applicants have found that wash-off personal wash compositions comprising oil provide enhanced measured/perceived lightening relative to those which do not contain oil.

As far as applicants are aware, the linkage between harshness (as measured, for example, by zein testing) and the effect this has on skin lightening is unknown. Furthermore a method of enhancing skin lightness, in relative terms, by utilizing a composition which is less harsh relative to a first, harsher composition is also believed to be novel.

### BRIEF DESCRIPTION OF THE FIGURES

FIG. 1 (measuring the lateral, or outer side, of the forearm) and FIG. 2 (measuring the volar, or inner side, of the forearm) indicate the lightening effect of a mild synthetic surfactant formulation (e.g., a composition comprising 13% acylaminopolyglycol ester sulfate and 7% cocoamidopropyl betaine and having a zein solubility about 2% or less, more specifically about 0.7%) whose zein solubility is of lesser value relative to a comparative “harsher”, liquid soap formulation (e.g., composition comprising various fatty acid soaps and having zein value of 4%). The lightening effect was measured by trained assessors (FIG. 1) or by subjects themselves (FIG. 2).

FIGS. 3 and 4 again measure the lightening effect of mild synthetic formulation versus a soap formulation as well as comparing harsh syndet versus soap. The effect was measured by trained assessors (FIG. 3) or by subjects themselves (FIG. 4).

FIG. 5 measures the lightening effect of mild synthetic composition with oil (lighter) versus one without, as measured by trained assessors.

FIG. 6 measures lightening effect of mild synthetic compositions with oil versus one without, as measured by the subjects themselves.

FIG. 7 measures lightening effect of a mild soap-like formulation (e.g., containing fatty acids and carboxylate surfactant to make less harsh as measured by zein) versus harsher soap formulation, as measured by trained assessors.

FIG. 8 measures lightening effect of milder soap-like formulation versus soap as measured by subjects themselves.

FIG. 9 is summary of zein dissolution for the various formulations.

### DETAILED DESCRIPTION OF THE INVENTION

The present invention relates to a method of enhancing measured or perceived skin lightening effect of a wash-off, personal product composition relative to a second wash-off personal product composition. The effect is measured by individuals using the compositions and/or by trained evaluators. Unexpectedly, applicants have found there is a correlation between the zein solubility of a composition (relative to a zein solubility of a comparative composition) and the skin lightening effect of the composition.

Specifically, when the first composition has a zein solubility (as measured by procedure defined herein) of about 2% or less and a second composition has a zein solubility greater than that of the first composition, preferably greater than about 3% in absolute value, applicants have demonstrated that the first composition is perceived (measurably perceived) to have a greater skin lightening effect relative to the second.

Measurement Of Skin Lightening

As will be discussed in more detail below, the measurement for skin lightening is a perceived effect. What this means is that assessment of lightness or darkness of skin is made either as a self assessment or by a trained evaluator. In either case, the assessor makes a judgement on a scale of plus 4 (+4) to minus 4 (−4) from which the assessor is asked to assess color fundamentals in two tested arms (one treated with “harsher” product and one with “less harsh” product) ranging from “strongly lighter” (+4) to strongly darker in color (−4).

Products

The products of the invention are wash-off personal product compositions. The compositions may be delivered, for example, in the form of liquid cleansers and body washes, bars, and non-wovens.

In one embodiment, the cleanser is in the form of a liquid cleanser or body wash. Generally, although not necessarily, such liquid cleansing compositions will comprise at least one surfactant. Typically, the compositions comprises 1% to 99% by wt., preferably 2% to 85%, more preferably 3% to 50% by wt. of a mild surfactant system. Since applicants have discovered a correlation between mildness and lightening, it is an important aspect of the invention that zein solubilization of the composition, whether a single surfactant, a combination of surfactants in a surfactant system or no surfactant at all are used, be less than the zein solubilization of a second composition to which it is compared. Preferably, zein solubilization of compositions of the invention should be about 2% or less, as measured by tests noted below.

If a surfactant is used, a number of anionic, nonionic, cationic, zwitterionic and/or amphoteric surfactants may be used, alone or in combination, as long as, as noted, the compositions maintain an overall zein solubility less than that of the composition against which they are compared, preferably about 2 and below.

Among suitable anionic actives which may be used are the alkyl ether sulfates, acyl isethionates, alkyl ether sulfonates, sarcosinates, sulfosuccinates, taurates and combinations thereof. Among suitable amphoteric actives may be included alkylbetaines, amidopropyl betaines, amidopropyl sultaines and combinations thereof.

Alkyl ether sulfates of the present invention will be of the general formula

R−(OCH₂CH₂)ₙOSO₃−M⁺

wherein R ranges from C₈-C₂₀ alkyl, preferably C₁₂-C₁₅ alkyl, n is an integer from 1 to 40, preferably from 2 to 9, optimally about 3, and M⁺is a sodium, potassium, ammonium or triethanolammonium cation.

Typical commercial co-actives of this variety are listed in the Table below:

| Trademark | Chemical Name | Physical Form | Manufacturer |
|---|---|---|---|
| Steol | Sodium Laureth | Liquid | Stepan |
| CS 330 | Sulfate | | |
| Standopol | Sodium Laureth | Liquid | Henkel |
| ES-3 | Sulfate | | |
| Alkasurf | Sodium Laureth | Paste | Alkaril |
| ES-60 | Sulfate | | |
| Cycloryl | TEA Laureth | Paste | Cyclo |
| TD | Sulfate | | |
| Standopol | Sodium Laureth-12 | Liquid | Henkel |
| 125-E | Sulfate | | |
| Cedepal | Sodium Trideceth | Paste | Miranol |
| TD407MF | Sulfate | | |
| Standopol | Ammonium Laureth | Liquid | Henkel |
| EA-2 | Sulfate | | |

Alkyl ether sulfonates may also be employed for the present invention. Illustrative of this category is a commercial product known as Avenel S-150 commonly known as a sodium C₁₂-C₁₅ Pareth-15 sulfonate.

Another active type suitable for use in the present invention is that of the sulfosuccinates. This category is best represented by the monoalkyl sulfosuccinates having the formula R₂OCCH₂CH(SO₃—Na+)COO-M+; and amido-MEA sulfosuccinates of the formula: RCONHCH₂CH₂O₂CCH₂CH(SO₃-M⁺)COO-M⁺; wherein R ranges from C₈-C₂₀ alkyl, preferably C₁₂-C₁₅ alkyl and M⁺ is a sodium, potassium, ammonium or triethanolammonium cation. Typical commercial products representative of these co-actives are those listed in the Table below:

| Trademark | Chemical Name | Physical Form | Manufacturer |
|---|---|---|---|
| Emcol 4400-1 | Disodium Lauryl | Solid | Witco |
| | Sulfosuccinate | | |
| Witco C5690 | Disodium | Liquid | Witco |
| | Cocoamido MEA | | |
| | Sulfosuccinate | | |
| McIntyre | Disodium | Liquid | McIntyre |
| Mackanate | Cocoamido MEA | | |
| CM40F | Sulfosuccinate | | |
| Schercopol | Disodium | Liquid | Scher |
| CMSNa | Cocoamido MEA | | |
| | Sulfosuccinate | | |
| Emcol 41OOM | Disodium | Paste | Witco |
| | Myristamido MEA | | |
| | Sulfosuccinate | | |
| Schercopol | Disodium | Liquid | Scher |
| | Oleamido MEA | | |
| Varsulf | Disodium | Solid | Scherex |
| S13333 | Ricionoleamido | | |
| | MEA Sulfosuccinate | | |

Sarcosinates may also be useful in the present invention as a co-active. This category is indicated by the general formula RCON(CH₃)CH₂CO₂-M⁺, wherein R ranges from C₈-C₂₀ alkyl, preferably C₁₂-C₁₅ alkyl and M⁺ is a sodium, potassium ammonium or triethanolammonium cation. Typical commercial products representative of these co-actives are those listed in the Table below:

| Trademark | Chemical Name | Physical Form | Manufacturer |
|---|---|---|---|
| Hamposyl L-95 | Sodium Lauroyl | Solid | W. R. Grace |
| | Sarcosinate | | |
| Hamposyl TOC-30 | TEA Cocoyl/ | Liquid | W. R. Grace |
| | Sarcosinate | | |

Taurates may also be employed in the present invention as co-actives. These materials are generally identified by the formula RCONR′CH₂CH₂SO₃-M⁺, wherein R ranges from C₈-C₂₀ alkyl, preferably C₁₂-C₁₅ alkyl, R′ ranges from C₁-C₄ alkyl, and M⁺ is a sodium, potassium, ammonium or triethanolammonium cation. Typical commercial products representative of these co-actives are those listed in the Table below:

| Trademark | Chemical Name | Physical Form | Manufacturer |
|---|---|---|---|
| Igepon TC 42 | Sodium Methyl | Paste | GAF |
| | Cocoyl Taurate | | |
| Igepon T-77 | Sodium Methyl | Paste | GAF |
| | Oleoyl Taurate | | |

Within the category of amphoterics there are three general categories suitable for the present invention. These include alkylbetaines of the formula RN⁺(CH₃)₂CH₂CO₂-M³⁰ , amidopropyl betaines of the formula RCONHCH₂CH₂CH₂N⁺(CH₃)₂CH₂CO₂-M⁺, and amidopropyl sultaines of the formula RCONHCH₂CH₂N⁺(CH₃)₂CH₂SO₃-M⁺ wherein R ranges from C₈-C₂₀ alkyl, preferably C₁₂-C₁₅ alkyl, and M⁺ is a sodium, potassium, ammonium or triethanolammonium cation. Typical commercial products representative of these co-actives are found in the Table below:

| Trademark | Chemical Name | Physical Form | Manufacturer |
|---|---|---|---|
| Tegobetaine F | Cocamidopropyl | Liquid | Goldschmidt |
| | Betaine | | |
| Lonzaine C | Cocamidopropyl | Liquid | Lonza |
| | Betaine | | |
| Lonzaine CS | Cocamidopropyl | Liquid | Lonza |
| | Hydroxysultaine | | |
| Lonzaine 12C | Coco-Betaine | Liquid | Lonza |
| Schercotaine | Myristamidopropyl | Liquid | Lonza |
| MAB | Betaine | | |
| Velvetex | Oleyl Betaine | Paste | Henkel |
| OLB-50 | | | |

Within the broad category of liquid actives, the most effective are the alkyl sulfates, alkyl ether sulfates, alkyl ether sulfonates, sulfosuccinates, and amidopropyl betaines.

Another preferred surfactant is an acyl isethionate having the formula

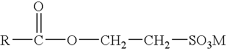

in which R denotes a linear or branched alkyl group and M denotes an alkali metal or alkaline earth metal or an amine.

Another surfactant which may be used are the monoalkyl or dialkylphosphate surfactants.

Another mild surfactant which may be used, preferably used as primary surfactant in combination with other surfactants noted above, is sodium coco glyceryl ether sulfonate. While desirable to use because of its mildness properties, this coco AGS alone does not provide optimum lather creaminess. A sodium 90/10 coconut/tallow alkyl AGS distribution is preferred for creaminess. Salts other than the sodium salt such as TEA-, ammonium, and K-AGS and chain length distributions other than 90/10 coconut/tallow are usable at moderate levels. Also, some soap may be added to improve lather volume and speed of lathering. Certain secondary co-surfactants used in combination with AGS can also provide a creamier and more stable lather. These secondary surfactants should also be intrinsically mild. One secondary surfactant that has been found to be especially desirable is sodium lauroyl sarcosinate (trade name Hamposyl L, made by Hampshire Chemical).

The amphoteric betaines and sultaines noted above can be used as the sole surfactant, but are more preferred as a co-surfactant. Nonionics generally should not be used as the sole surfactant in this product if high foaming is desirable; however, they can be incorporated as a co-surfactant.

Nonionic and cationic surfactants which may be used include any one of those described in U.S. Pat. No. 3,761,418 to Parran, Jr., hereby incorporated by reference into the subject application. Also included are the aldobionamides as taught in U.S. Pat. No. 5,389,279 to Au et al; and the polyhydroxy fatty acid amides as taught in U.S. Pat. No. 5,312,934 to Letton, both of which are incorporated by reference into the subject application.

Soaps may be used at levels of about 0.1 to 10%. Soaps can be used at higher level (for example, to provide soap-like feel) provided that the overall surfactant mixture is milder than soap and retains zein values within the scope of the invention. The soaps may be added neat or made in situ via adding a base, e.g., NaOH; to convert free fatty acids.

Of course, as noted above, soaps should only be used as cosurfactants to the extent that zein solubilization of composition is less (milder) than a composition to which compared, preferably about 2 and below.

A preferred surfactant active system is one such that acyl isethionate comprises 1 to 15% by weight of the total composition and/or an anionic other than acyl isethionate (e.g., ammonium lauryl ether sulfate) comprises 1 to 15% by weight of the total composition and amphoteric comprises 0.5 to 15% by weight of the total composition.

Another preferred active system is one comprising 1 to 20% alkyl ether sulfate. Preferred surfactant active systems may also contain 1 to 10% alkali metal lauryl sulfate or C₁₄-C₁₆ olefin sulfonate instead of acyl isethionate.

The cleansing products may be structured using, for example, external structurants such as cross-linked polyacrylates and/or clays or they may be structured with other components (e.g., unsaturated and/or branched long chain C₈ to C₂₄ liquid fatty acid or ester derivatives) which would cause the liquid to have a “lamellar” structure as described, for example, in U.S. Pat. No. 5,952,286.

The liquid products may have other ingredients typically found in liquid cleansers. Among these are included (without limitation) auxiliary thickeners (e.g., carboxymethyl cellulose, hydroxyethylcellulose); perfumes; sequestering agents (e.g., ethyl diamine tetra acetate, known as EDTA); coloring agents; opacifiers and pearlizers (e.g., zinc or magnesium stearate, titanium dioxide).

Other optionals include antimicrobial agents; preservatives (e.g., parabens, sorbic acid); suds boosters (e.g., coconut acyl mono- or diethanolamide); antioxidants; cationic conditioners (e.g., Merquat® and Jaguar® type conditioners); polyalkylene glycols, glycerin and other water-soluble conditioning agents; thickeners; exfoliates; ionizing salts; organic acids (e.g., citric or lactic acid).

In a preferred embodiment of the invention, the compositions may comprise on oil/emollient.

Among oils which can be used are:

| | |
|---|---|
| Vegetable oils: | Examples include orachis oil, |
| | castor oil, cocoa butter, |
| | coconut oil, corn oil, cotton |
| | seed oil, olive oil, palm kernel oil, |
| | rapeseed oil, safflower seed and |
| | sesame seed oil, soybean |
| | oil. |
| Ester: | Examples include butyl myristate, |
| | cetyl palmitate, glyceryl |
| | stearate or isostearate, isobutyl, |
| | palmitate, isopropyl |
| | isostearate, isopropyl myristate. |

Other classes of oil/emollient include animal fats (lanolin, lard), fatty acids and alcohols (behenic acid, palmitic acid, stearic acids, cetyl alcohol).

Further are included mineral oil, petrolatum, silicone oil.

It has been found that, in otherwise identical compositions, use of oil/emollient leads to enhanced skin lightening effect as measured by trained evaluators and or self-assessment by test subjects.

In another embodiment, the compositions may be delivered in the form of a bar composition.

Bar compositions may comprise 5% to 90% by wt. of a surfactant wherein the surfactants may be any of the surfactants noted above (e.g., anionics, nonionics, amphoteric/zwitterionics, cationics) or mixtures thereof. As with liquid cleansers noted above, the amount of pure soap actually used is preferably not too high as to effect overall harshness of the bar (i.e., zein values of bar compositions are still within the scope of the invention).

The bar compositions also comprises 0.1 to 20% water, preferably 1 to 15% by wt. water.

The bar composition further comprises 0.1 to 80% by wt., preferably 5% to 75% by wt. of a structuring aid and/or inert filler. Such structurants can be used to enhance the bar integrity, improve the processing properties, and enhance desired user sensory profiles.

The structurant is generally long chain, preferably straight and saturated, (C₈-C₂₄) fatty acid or ester derivative thereof; and/or branched long chain, preferably straight and saturated, (C₈-C₂₄) alcohol or ether derivatives thereof.

A preferred bar structurant is polyalkylene glycol with molecular weight between 2000 and 20,000, preferably between 3000 and 10,000. Those PEGs are commercially available, such as those marketed under the tradename of CARBOWAX SENTRY PEG8000^{(R)} or PEG4000^{(R)} by Union Carbide.

Other ingredients that can be used as structurants or fillers include starches, preferably water soluble starches such as maltodextrin and polyethylene wax or paraffin wax.

Structuring aids can also be selected from water soluble polymers chemically modified with hydrophobic moiety or moieties, for example, EO-PO block copolymer, hydrophobically modified PEGs such as POE(200-glyceryl-stearate, glucam DOE 120 (PEG Methyl Glucose Dioleate), and Hodg CSA-102 (PEG-150 stearate), and Rewoderm^{(R)} (PEG modified glyceryl cocoate, palmate or tallowate) from Rewo Chemicals.

Other structuring aids which may be used include Amerchol Polymer HM 1500 (Nonoxynyl Hydroethyl Cellulose).

In addition, the bar compositions of the invention may include 0 to 15% by wt. optional ingredients as follows:

perfumes; sequestering agents, such as tetrasodium ethylenediaminetetraacetate (EDTA), EHDP or mixtures in an amount of 0.01 to 1%, preferably 0.01 to 0.05%; and coloring agents, opacifiers and pearlizers such as zinc stearate, magnesium stearate, TiO₂, EGMS (ethylene glycol monostearate) or Lytron 621 (Styrene/Acrylate copolymer); all of which are useful in enhancing the appearance or cosmetic properties of the product.

The compositions may further comprise antimicrobials such as 2-hydroxy-4,2′4′ trichlorodiphenylether (DP300); preservatives such as dimethyloldimethylhydantoin (Glydant XL1000), parabens, sorbic acid etc.

The compositions may also comprise coconut acyl mono- or diethanol amides as suds boosters, and strongly ionizing salts such as sodium chloride and sodium sulfate may also be used to advantage.

Antioxidants such as, for example, butylated hydroxytoluene (BHT) may be used advantageously in amounts of about 0.01% or higher if appropriate.

Cationic polymers as conditioners which may be used include Quatrisoft LM-200 Polyquaternium-24, Merquat Plus 3330-Polyquaternium 39; and Jaguar^{(R)} type conditioners.

Polyethylene glycols as conditioners which may be used include:

| | | |
|---|---|---|
| Polyox | WSR-205 | PEG 14M, |
| Polyox | WSR-N-60K | PEG 45M, or |
| Polyox | WSR-N-750 | PEG 7M. |

Another ingredient which may be included are exfoliants such as polyoxyethylene beads, walnut shells and apricot seeds.

Zein Testing

The criticality of the invention, as noted above, is not in any particular combination of surfactants or other composition components, but in the zein solubilization measurement of each composition.

The primary compositions of the invention preferably have a zein solubility of about 2% or less.

According to the method of the invention, lightening effect is noted when comparing a first composition (which generally has zein solubilization of 2% or less) relative to second wash-off composition, wherein said second composition has zein solubility greater than that of the first composition.

Thus, while it is preferred that the second comparative composition have a zein solubility greater than 3% in absolute value, it should be understood that, if the first composition has for example, zein solubility of 1%, the second composition need have only a solubility greater than 1%. That is, lightening effect can be observed based on selective differences in harshness alone.

Except in the operating and comparative examples, or where otherwise explicitly indicated, all numbers in this description indicating amounts or ratios of materials or conditions or reaction, physical properties of materials and/or use are to be understood as modified by the word “about”.

Where used in the specification, the term “comprising” is intended to include the presence of stated features, integers, steps, components, but not to preclude the presence or addition of one or more features, integers, steps, components or groups thereof.

The following examples are intended to further illustrate the invention and are not intended to limit the invention in any way.

Unless indicated otherwise, all percentages are intended to be percentages by weight.

### EXAMPLES

Protocol for Testing Perceived Skin Lightening

Typical Study Design

A typical skin lightening clinical study is carried out for anywhere from two to eight weeks. In a typical study, subjects washed their forearms with the assigned test products at home twice daily with a minimum of 6 hours between washes. Subjects came to the test center at week 0 (baseline), 1, 4, 6, and 8. Subjects were instructed not to wash their forearms with the test products in the morning on the days of the visit to the test center. At each visit, experts conducted a visual assessment for skin color and safety assessment (erythema) on the forearms, and, in addition, the subject conducted a self assessment of skin color. Subjects then washed their forearms with the test products in the presence of study personnel to confirm that proper application technique was being used. Test product weights were taken at each visit in the test center to ensure the appropriate amount of test material was used.

Study Population

Seventy subjects were typically recruited to ensure a minimum of 60 subjects per cell completed the study. Inclusion and exclusion criteria for panelists was typical as set forth below for studies of this invention:

Inclusion Criteria: Chosen subjects were females between 18-35 years old, of Indonesian decent, having a skin color grade minimum of 6 from 1 to 9 color skin shade point scale (where 1 is lightest and 9 is darkest). Subjects had reasonably uniform forearm skin which is pigmented darker than sun protected skin, and agreed to maintain normal sun exposure (between 30 minutes to 90 minutes per day) and to wear short sleeve shirts during daytime hours.

Exclusion Criteria: Subject could not have a history of photosensitivity or a known allergy to soap, detergent and sunscreen. Subject must not currently be using or have used a skin lightening product in the past 12 months.

Method of Product Application

Subjects moistened one of their forearms (both lateral and volar) with water. Subjects used 1 mL of test product with 5 mL water to generate lather on their wet palm and gently washed their lateral and volar forearm (from elbow to wrist) for 30 seconds. Subjects allowed the lather to remain on the forearm for 10 seconds. The subject carefully rinsed the forearm with water for at least 10 seconds and then gently dried with towel. The forearm was washed twice daily (morning and evening) for the 8-week study period. The time between application was at least 6 hours. A product application diary was maintained by the subjects and reviewed by study personnel at each visit.

At each visit to the test center, subjects washed their forearm with the test product in the presence of study personnel to confirm that proper application technique was being used. Test product weights were taken at each visit in the test center to ensure the appropriate amount of test material is being used.

Clinical Evaluation Methods

Visual Assessment

Visual assessment of the volar (inner) and lateral (outer) forearm was conducted at week 0 (baseline), 1, 4, 6, and 8. On days of visual evaluation, subjects did not wash their forearm with the test products in the morning, or allowed at least 6 hours after application to elapse prior to the evaluation.

All visual evaluations were conducted under consistent lighting conditions, which consisted of fluorescent light bulbs. A black velvet background cloth was placed over the front of the subject but under the arms for all visual evaluations.

a. Subject Self Assessment Test

At visits 0, 1, 4, 6 and 8 the subject was asked to compare their right forearm with their left forearm for differences of skin color (both lateral and volar separately).

The following directed difference questions were asked:

Is the RIGHT arm lighter or darker than the LEFT arm?

If so by how much? Assessment was graded according to the following scale:

| | |
|---|---|
| +4 | strongly lighter in color |
| +3 | markedly lighter in color |
| +2 | moderately lighter in color |
| +1 | slightly lighter in color |
| 0 | no difference in color |
| −1 | slightly darker in color |
| −2 | moderately darker in color |
| −3 | markedly darker in color |
| −4 | strongly darker in color |

b. Visual Directed Difference by Trained Evaluator

The right forearm was compared to the left forearm for tone and clarity differences (both lateral and volar separately) by evaluator.

Again, the following directed difference questions were asked:

Is the RIGHT arm lighter or darker than the LEFT arm?

If so by how much? Again, assessment was graded according to the following scale:

| | |
|---|---|
| +4 | strongly lighter in color |
| +3 | markedly lighter in color |
| +2 | moderately lighter in color |
| +1 | slightly lighter in color |
| 0 | no difference in color |
| −1 | slightly darker in color |
| −2 | moderately darker in color |
| −3 | markedly darker in color |
| −4 | strongly darker in color |

Zein Solubility Measurement

Zein solubility was used to compare the mildness of cleansers. The test protocol is described below:

1. 5 g of the tested cleanser was mixed with 45 g of deionized water using a magnetic stirrer for 5 to 10 minutes to form a uniform solution.

2. 5 ml of the solution was withdrawn and filtered through 0.45 micron filter into a vial for % solid measurement (the solution was marked as blank).

3. About 2 g of zein was added to the remaining solution and mixed for 60 minutes using a magnetic stirrer. The solution was shaken often to ensure that there was enough undissolved zein in the mixture. If most of the zein dissolved, 1 more gram of zein was added into the solution and mixing was continued (zein excess was always kept, but not too much because zein will swell and make the solution difficult to filter).

4. After mixing, 5 ml of the supernatant was withdrawn in a syringe, filtered through 0.45 micron filter into a vial, and this was marked as sample. The solution was centrifuged at 3,000 rpm for 5 minutes before filtration if the supernatant was hard to separate.

5. The % solid of the blank and the sample was determined by weighing about 3 to 4 grams of the filtrate in an aluminum dish using an analytical balance and drying the filtrate overnight in a 75° C. oven.

6. The % of solid zein dissolved in the diluted liquid solution was calculated using the following equation.

% solid of zein solubilized=% solid of sample 1-% solid of blank.

### Example 1

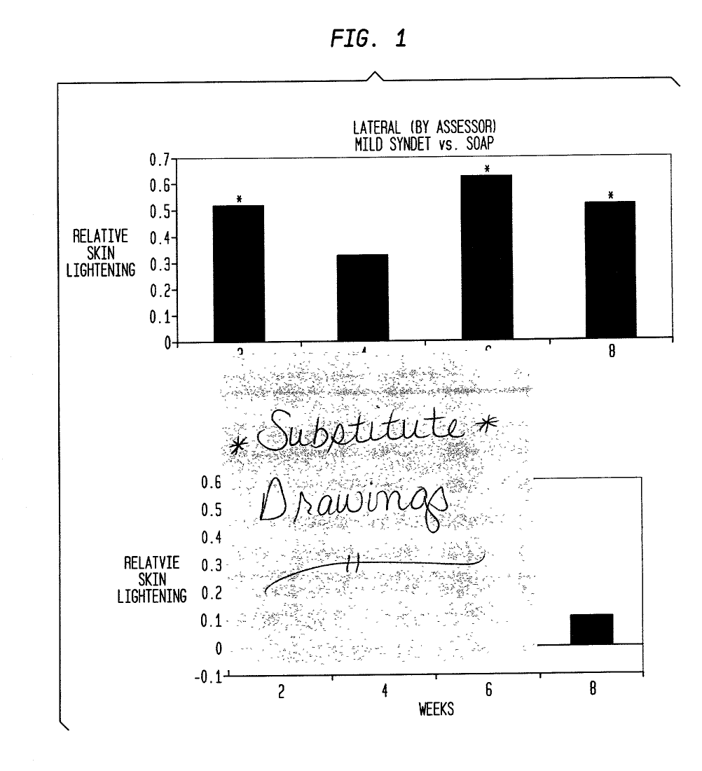

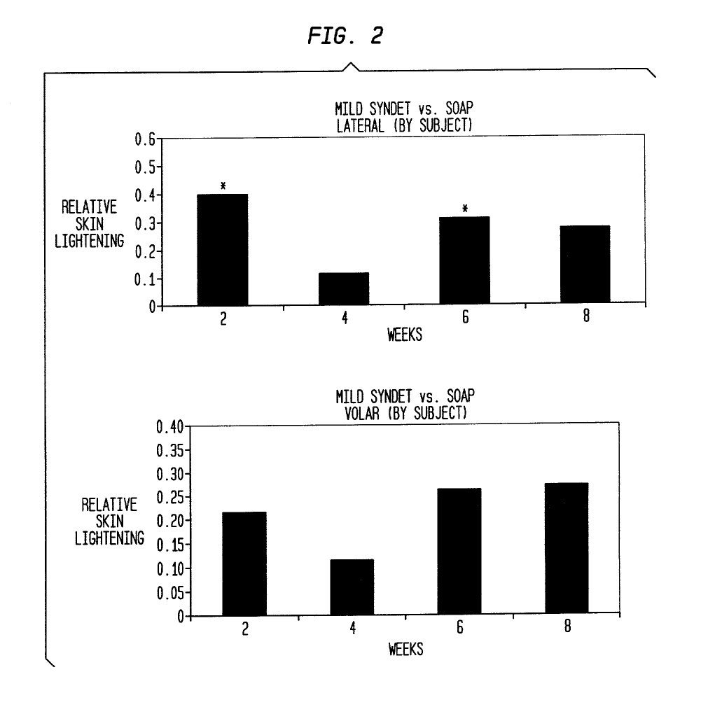

In order to show changes in skin lightening based on differences in formulation (and generally on harshness differences as measured by zein dissolution test noted above), applicant tested “visual directed difference” on both lateral and volar parts of forearm, both measured by trained assessor (FIG. 1) and by self assessment (FIG. 2). The tests were to measure difference between a mild synthetic surfactant formulation (having zein dissolution value of about 0.7) as set forth in Table 1 below and a soap product (having zein dissolution value of about 4) as set forth in Table 2 below:

TABLE 1
Mild Synthetic

| FULL CHEMICAL NAME | % ACTIVE LEVEL IN |
|---|---|
| OR CTFA NAME | FORMULATION |
| Acylaminopolyglycol | 13.0 |
| Ether Sulfate | |
| Cocoamido Propyl Betaine | 5.0-8.0 |
| Sunflower Seed Oil | 5.0-7.0 |
| Petrolatum | 5.0-8.0 |
| Glycerin | 5.0-8.0 |
| Guar Hydroxypropyl | 1.0-2.0 |
| Trimonium Chloride | |
| Ethylene Diamine | 1.0-3.0 |
| Tetraacetic Acid | |
| Fragrance | 1.0 |
| DMDM Hydantoin/ | 0.20 |
| Iodoproynyl Butylcarbamate | |
| Deionized Water | To 100 |

TABLE 2
Soap

| CHEMICAL NAME | % W/W |
|---|---|
| Lauric Acid | 13 |
| Myristic Acid | ˜7.0 |
| Potassium Hydroxide | 6-6.5 |
| Glycerol | 2-4 |
| Ethylene Glycol Monostearate | 2-5 |
| Stearic Acid | 1.0 |
| Hydroxy Ethyl Cellulose | 0.9 |
| Citric Acid | 0.2 |
| Imidazolidinil urea | 0.2 |
| Tetrasodium EDTA | 0.1 |
| Hydrolized Milk Protein | 0.05 |
| Perfume | 0.1-3 |
| Water | To 100 |

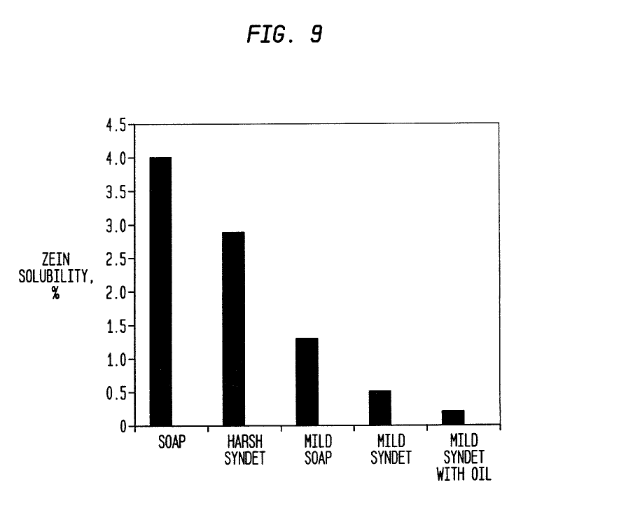

As seen in FIG. 9, the mild syndet dissolves far less zein (about 0.7%) than “soap” (about 4% zein dissolved) and certainly dissolves at a level of “about 2% and below” as per the preferred embodiments of the invention.

FIG. 1 (top panel) is a visual directed difference test of the two products comparing lateral forearm as measured by a trained assessor. As this is a paired comparison, positive values indicate the benefit in lightening of mild synthetic composition (e.g., syndet) over soap over 8 week period as measured by assessor. As clearly seen in FIG. 1, a statistically significant difference is seen in 3 of the measured points (week 2, 6 and 8).

FIG. 1 (bottom panel) is the same test as for top panel of FIG. 1, but measured by an expert on the volar region (interior) of the forearm. Again, there is a statistical difference at least in week 6 and the trend for visual difference is seen throughout the test.

FIG. 2 (top and bottom) are same tests (for lateral and volar region of forearm), but measured by the panelists themselves. The top panel of FIG. 2 again shows statistical significance in at least 2 measured points and the bottom patel shows strongly observable difference as well.

### Example 2

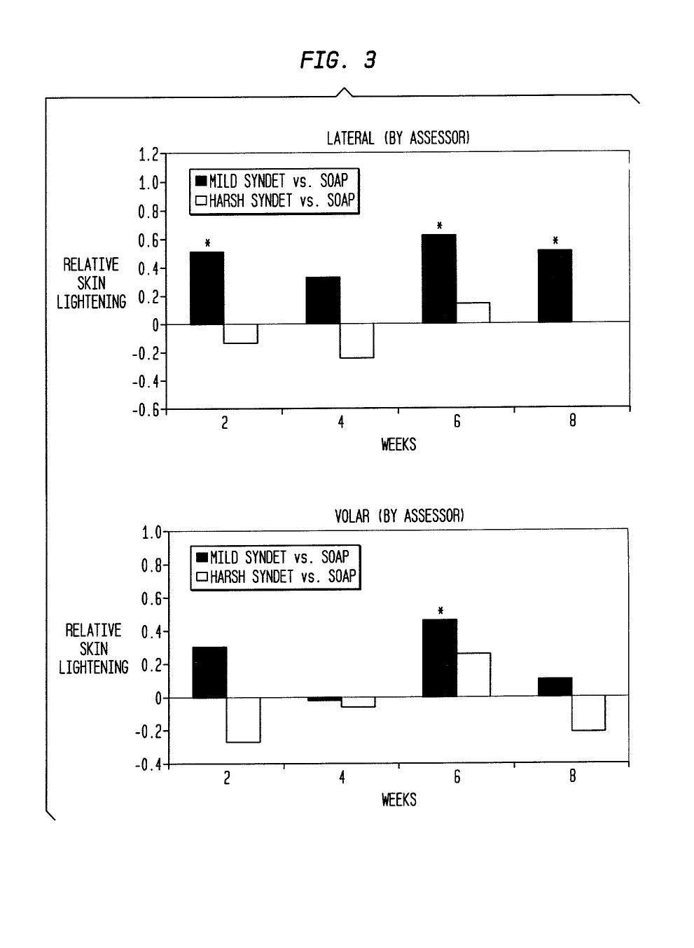

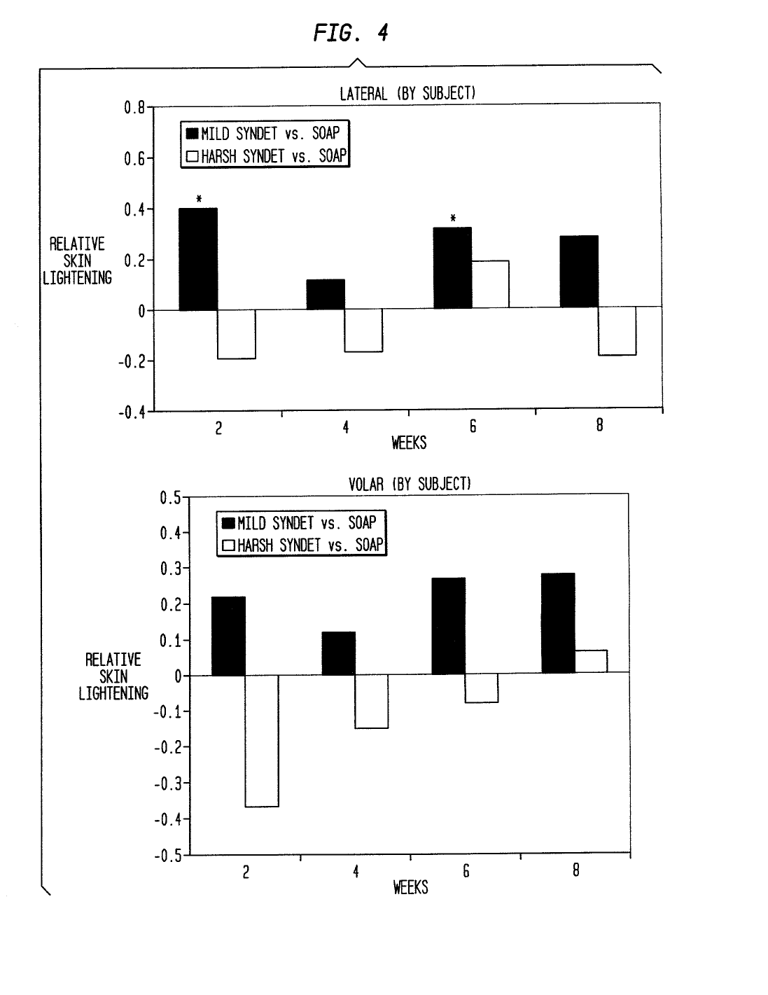

FIGS. 3 and 4 were designed to show differences in lightening between a mild synthetic liquid composition (i.e., same composition of Table 1 in Example 1) and a “harsh” synthetic liquid composition as set forth in Table 3 below:

TABLE 3
Harsh Synthetic

| | % ACTIVE IN |
|---|---|
| CFTA/CHEMICAL NAME | FORMULATION |
| Ammonium Lauryl-2-Sulphate | 16-20 |
| Potassium Stearate | 1.00 |
| Ethylene Glycol Monostearate | 3.50 |
| Glycerine | 6-10 |
| Niacinamide (Vitamin B3) | 2.00 |
| Formaldehyde | .05-2.0 |
| Lactic Acid | 05.-1.0 |
| EDTA | 0.10 |
| NaCl | 2.00 |
| Perfume | 0.70 |
| Water | To 100.00 |

FIG. 3 was measured by trained assessor and FIG. 4 by subjects themselves.

As seen in FIG. 9, the harsh syndet has zein dissolution of about 2.8 compared to 0.7 for mild syndet.

FIGS. 3 and 4 are not a direct comparison of mild syndet versus harsh but instead shows effect of mild syndet versus soap (on left side of each measured period) compared to effect of harsh syndet versus soap (on right side of each measured period). This indirect comparison clearly shows that mild syndet, which has far lower zein dissolution value, has far greater perceived lightening than the harsh syndet. Thus, lightening is clearly a function of composition harshness.

### Example 3

### Mild Syndet With and Without Moisturizer

Another aspect of the subject invention is that perceived lightening is enhanced even further depending on whether or not moisturizing oil is used.

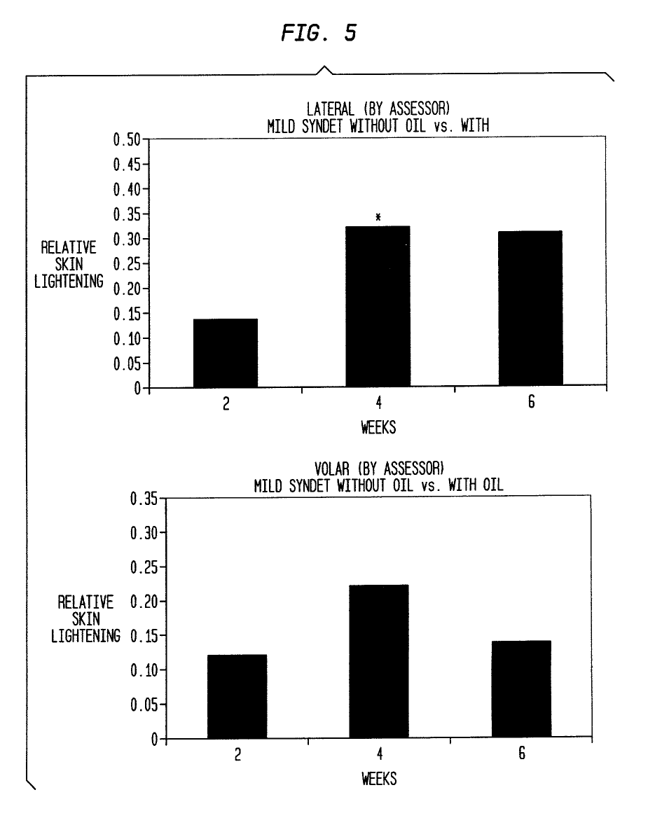

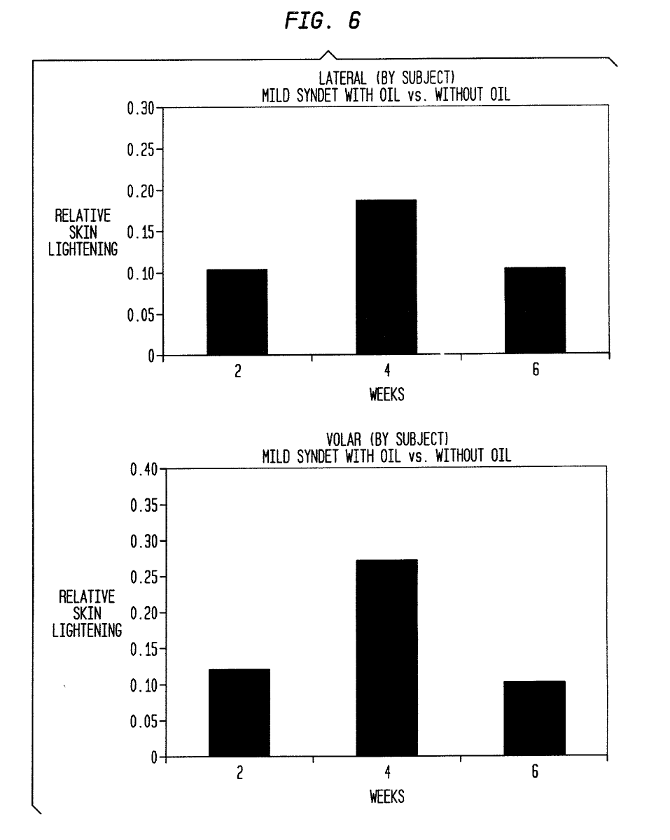

In FIGS. 5 and 6, this is shown based on comparison of mild synthetic liquid (with moisturizing oil) of Table 1 to mild liquid composition with no moisturizing oil having formulation set forth in Table 4 below:

TABLE 4
Mild Syndet Formulation Without Moisturizing Oil

| FULL CHEMICAL NAME OR | % ACTIVE LEVEL IN |
|---|---|
| CTFA NAME | FORMULATION |
| Acylaminopolyglycol ether sulfate | 13.00 |
| Cocoamido Propyl Betaine | 7.00 |
| Sunflower Seed oil | 0.00 |
| Petrolatum | 0.00 |
| Glycerin | 6.00 |
| Guar Hydroxypropyl trimonium Chloride | 1.60 |
| Ethylene diamine tetraacetic acid | 2.00 |
| FRAGRANCE | 1.00 |
| DMDM Hydantoin/Iodoproynyl | 0.20 |
| Butylcarbamate | |
| DI Water | To 100 |

FIGS. 5 and 6 clearly show that syndet with oil was perceived as lightening relative to syndet without oil.

### Example 4

Finally, in order to show that lightening effect is function of the harshness of the liquid and not simply whether or not it is a liquid with synthetic surfactant, applicants conducted test comprising harsh soap as set forth in Table 2 of Example 1 (having zein dissolution of about 4 as seen in “FIG. 14) to a mild “soap-like” (in its sensory attributes, for example, because of presence of basic acid) syndet (zein dissolution of about 1.4) as set forth in Table 5 below.

TABLE 5
Mild Soap-like Formulation

| FULL CHEMICAL NAME OR CTFA NAME | % IN PRODUCT |
|---|---|
| Lauric Acid | 1.5 |
| Triethanol Amine | 1.1 |
| Sodium Lauroamphoacetate | 10.5 |
| Potassium Cocoyl Glycinate | 0.45 |
| Potassium Cocoyl Glutamate | 1.275 |
| Xanthan Gum | 1.2 |
| Glycerin | 10 |
| Citric Acid | 0.5 |
| DMDM HYDANTOIN | 0.2 |
| Perfume | 0.2-0.6 |
| DI Water | To 100 |

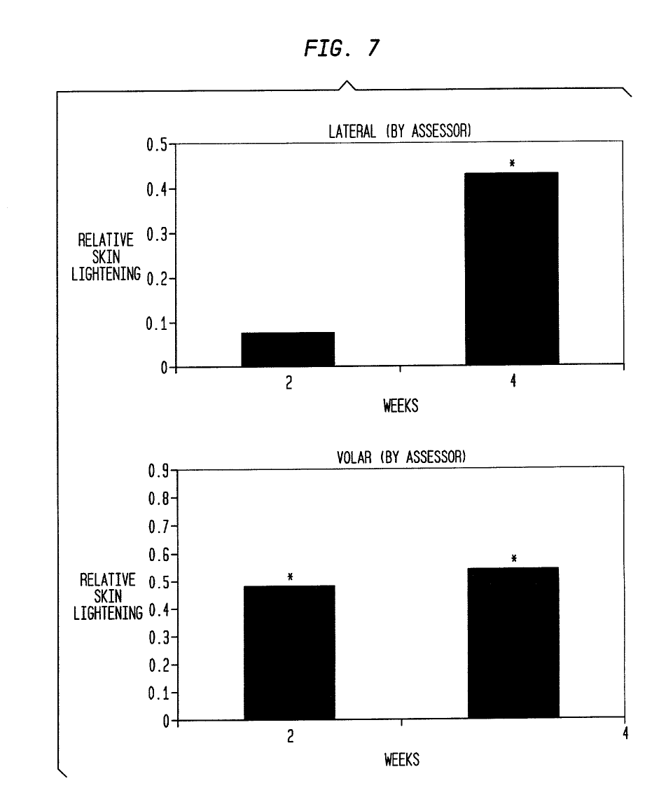

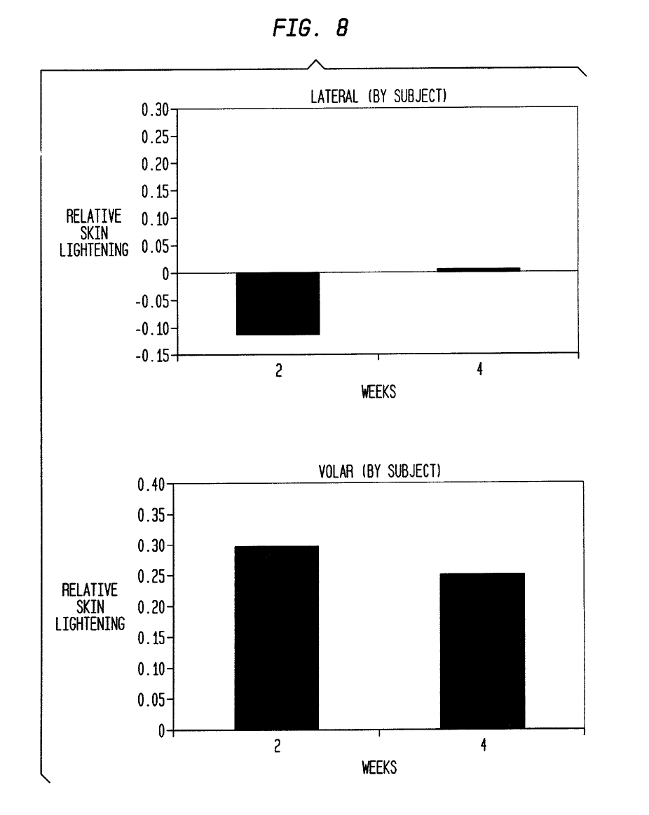

As noted from above and seen in FIG. 9, the “mild” soap-like formulation provides far lower zein dissolution values than regular “soap”. As seen in FIGS. 7 and 8, this mildness clearly correlates with enhanced perceived lightening, especially when measured by trained assessors (see FIG. 7).

1. A method for enhancing skin lightening of a first wash-off personal product composition relative to a second wash-off personal product composition which process comprises:
(a) utilizing as said first composition a composition which has a zein solubility of about 2% or less; and
(b) applying said first composition to skin;
wherein said lightening is measured relative to said second composition;
wherein said second composition has a zein solubility greater than the measured zein solubility of the first;
wherein said zein solubility is measured by calculating the percent solid of sample to be tested into which zein is dissolved and subtracting the percent solid of a blank of the same tested composition into which zein was not dissolved.

2. A method according to claim 1, wherein the zein solubility of the second composition for which zein solubility of first is compared is greater than about 3% in absolute value.

3. A method according to claim 1, wherein said first composition comprises a surfactant selected from the group consisting of anionic surfactant, nonionic surfactnt, amphoteric surfactant, zwitterionic surfactant, cationic surfactant and mixtures thereof.

4. A method according to claim 1 wherein percent solid of tested sample into which zein was dissolved was calculated by:
(a) adding zein to tested solution and mixing;
(b) withdrawing supernatant after mixing;
(c) optionally centrifuging and obtaining filtrate by filtering; and
(d) obtaining percent solid by drying and weighing filtrate.

5. A method according to claim 1, wherein said first composition is delivered in the form of a liquid composition.

6. A method according to claim 1, wherein said first composition is delivered in the form of a bar composition.

7. A method according to claim 1, wherein said first composition is delivered from a non-woven substrate.
